(19) United States
(12) Patent Application Publication
(10) Pub. No.: US 2007/0231258 A1
(43) Pub. Date: Oct. 4, 2007

(54) Peptide conjugates

(75) Inventors: Hannu Perakyla, Helsinki (FI); Merja Huttunen, Helsinki (FI); Eeva-Kaisa Karisalmi, Helsinki (FI); Mathias Bergman, Helsinki (FI); Hannu Elo, Helsinki (FI)

Correspondence Address: BUCHANAN, INGERSOLL & ROONEY PC, POST OFFICE BOX 1404, ALEXANDRIA, VA, 22313-1404, US

(73) Assignee: KARYON CTT LTD., Helsinki (FI)

(21) Appl. No.: 11/730,294

(22) Filed: Mar. 30, 2007

Related U.S. Application Data
(60) Provisional application No. 60/787,493, filed on Mar. 31, 2006.

(30) Foreign Application Priority Data
Mar. 31, 2006 (FI) 20065208

(51) Int. Cl. A61K 51/00; C07K 14/80

(52) U.S. Cl. 424/1.69; 530/400

(57) ABSTRACT

The present invention relates to a method for synthesizing peptide conjugates comprising a functional peptide, cyclic by means of a lactam bridge; a sulfur-linker bound to said cyclic peptide, wherein said sulfur-linker comprises sulfur or a sulfur-reactive site. The invention further relates to a method for synthesizing peptide-effector conjugates. The invention also relates to peptide conjugates. Peptide conjugates according to the present invention have improved half-life and increased availability at the active site and they are useful in cell targeting and tumor diagnosis and therapy.

### FIELD OF THE INVENTION

The present invention relates to a method for synthesizing peptide conjugates comprising a cyclized functional peptide, and optionally linking an effector unit to said peptide site-specifically. The present invention also relates to improved peptide conjugates comprising a cyclized functional peptide.

Peptide conjugates according to the present invention have improved half-life and increased availability at site of action and they are useful in identification, detection and selection applications, clinical applications and especially in tumor diagnosis and therapy.

### BACKGROUND OF THE INVENTION

Certain peptides have the ability to bind to specific biological material, cell types, cell surface structures or receptors therein, or to penetrate biological membranes in cells or organs. Conjugating a functional agent, an effector unit, to such a peptide provides the agent with a targeting or transport ability. Such a functional peptide conjugate (herein also called peptide-effector conjugate) serves detection and therapeutic purposes and is useful in diagnosis, therapy or research, or makes a therapeutic agent, e.g. antimicrobial, antiviral or cytostatic agent available at the site of action.

A great variety of drugs, agents or other effector units (collectively herein called effector units) show very poor tissue or organ penetration and have poor pharmacodynamic properties and tissue distribution. By conjugating such effector units to a functional peptide to form a peptide-effector conjugate, their biological and pharmacological properties can be drastically altered and possibly improved.

Abnormal changes of living tissue at the molecular level are common to many diseases and physiological disorders. Functional peptides that are capable of targeting those alterations can be very useful in early detection or diagnosis of, e.g., vascular diseases, disorders of the nervous system and cancer. Such peptides may also be useful in therapy e.g. as targeted drug carriers.

Malignant tumors are one of the greatest health problems of humans as well as animals, being one of the most common causes of death, also among young individuals. Available methods of treating cancer are quite limited, in spite of intensive research efforts during several decades. Although curative treatment (usually surgery in combination with chemotherapy and/or radiotherapy) is sometimes possible, malignant tumors are still one of the most feared diseases of mankind. In fact, curative treatment is rarely accomplished if the disease is not diagnosed early. There are numerous publications disclosing peptides homing to different cell and tissue types. Some of these are claimed to be useful as cancer targeting peptides. Among the earliest identified homing peptides described are the integrin and NGR-receptor targeting peptides described by Ruoslahti et al., in e.g., U.S. Pat. No. 6,180,084. These peptides home to angiogenic vasculature and bind to the NGR-receptor. More recently published targeting peptides are described in e.g., international patent publication WO2004/031218A1.

Serious problems associated with the use of short peptides are caused by their short lifetime in the body, due to enzymatic or chemical disintegration. Disintegration of the peptide may change the therapeutic effect of the conjugate in a deleterious way, or give misleading diagnosis. The original function and benefits of the peptide and the peptide-effector conjugate might be lost if the peptide breaks down. Thus, there is a recognized need for improving the stability of functional peptides and peptide-effector conjugates.

There are alternative ways of improving stability and in vivo lifetime of functional peptides. One is to restrict the spatial arrangements (conformations) of the molecule by means of cyclization. Another is the introduction of non-natural amino acids, such as D-amino acids or β-amino acids. Cyclization of the peptide may enhance the function of the peptide. Any approach, however, is useful only if the functional property of the peptide is preserved.

One type of peptide cyclization occurring in nature is based on a disufide bond between two cysteine amino acids forming a cystine bridge. A cystine bridge may increase the stability against enzymatic attacks, compared to an uncyclized peptide. The disulfide bond in cystine is, however, among the chemically most reactive bonds in peptides, both in vivo and in vitro, and brings therefore chemical instability and complications to the synthesis and use of useful conjugates, including metal complexes, which is well known to a person skilled in the art. To make metal complexes in the presence of disulfide bonds is especially problematic. Moreover, cysteine is prone to loose its stereo configuration during peptide synthesis.

Thus any methods for the preparation of stable cyclic peptide conjugates that comprise one, or more steps in which the cyclic structure is prepared by formation of a cystine or other disulfide bridge, suffer from serious drawbacks.

There is thus a recognized need for improved methods of synthesizing cyclic peptide conjugates that do not comprise any steps of formation or provision of cystine bridge or other disulfide bridge for cyclization and wherein another kind of structurally adequate (cystine-bridge-mimicking) stable bridge is formed (synthesized).

A bridge based on a bond of the same type as between the amino acids in the peptide, i.e. a lactam bridge, is one of the best choices for a cystine-bridge-mimicking substitute bridge, in view of stability and economy in use of chemical resources. The term “mimicking” is used to describe geometric similarity, including stereo configuration. A known possibility is an amide-bonded side chain-to-side chain approach in order to connect a diamino acid with a dicarboxylic amino acid. But the necessary special reagents for that purpose are costly, and the steps needed in these methods may require difficult reaction conditions, which are unsuitable for regular, especially automated, solid phase peptide synthesis.

A further, desirable feature of improved functional peptides is the possibility to extend the peptide outside either terminus of the cyclized peptide sequence in order to couple functional agents or effector units to the peptide.

A desirable feature of improved methods for making peptide-effector conjugates is to achieve the possibility of making sets of such conjugates that have the same peptide but different effectors, or the same effector but different peptides. Such sets are valuable e.g. in medical methods where the same directing (targeting or transport) function is needed both in therapy and diagnosis or follow-up, or where different directing functions are needed for a diagnostic study and for making the optimal choice between such functional peptides.

The known methods for making such peptide-effector conjugates, especially conjugates of cyclic peptides, suffer from problems of being nonsite-specific, restrictive to the composition of the peptide, or are based on unstable bonds or are complicated and tedious to carry out.

Thus, there is still a recognized need for improved methods of synthesizing structurally well-defined peptide conjugates that can be used to combine various functional peptides with various effectors in an easy and cost-effective way. It is also desirable that automatic devices can be used in such a method.

### SUMMARY OF THE INVENTION

It is an object of the present invention to provide a method of synthesizing a peptide conjugate comprising at least one functional peptide and at least one sulfur-linker, wherein said method comprising the steps of:

a) providing a dicarboxylic amino acid having a protecting group at its side chain carboxylic group;
b) synthesizing a peptide with a pre-determined amino acid sequence to form a functional peptide using protection, which is orthogonal with respect to the protecting group in step a);
c) constructing a synthetic peptide comprising said functional peptide of step b), said dicarboxylic amino acid of step a) C-terminally located from said functional peptide, and an N-terminal amino acid having an optionally protected non-side chain amino group;
d) removing the optional non-side chain protecting group of said N-terminal amino acid and the protecting group of the side chain of said dicarboxylic amino acid;
e) rendering the synthetic peptide obtained in step d) cyclic by connecting said non-side chain amino group to the unprotected side chain of said dicarboxylic amino acid to form a cyclic peptide;
f) coupling a sulfur-linker, selected from the group consisting of thiols, thioethers, disulfides, sulfoxides, sulfones and thiolates, to a non-side chain carboxylic group of said dicarboxylic amino acid;

in which method said functional peptide is a peptide capable of penetrating biological membranes, organs or tissue or targeting and binding selectively to desired materials or tumor tissue and said peptide comprises 3-30 amino acids, except amino acids having a thiol group.

Another object of the present invention is to provide a peptide conjugate according to formula (I):

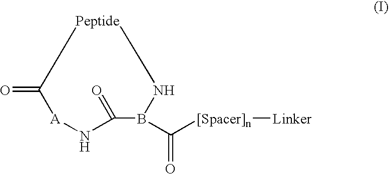

wherein

n is 0-1

“Peptide” is a functional peptide capable of penetrating biological membranes, organs or tissue or targeting and binding selectively to desired materials or tumor tissue and said peptide comprises 3-30 amino acids, except amino acids having a thiol group,

A is a residue of a D-amino acid or a β-amino acid,

B is a residue of a dicarboxylic amino acid,

“Spacer” is moiety providing distance between said sulfur-linker and said cyclic peptide and/or solubility enhancing properties to the peptide conjugate,

“Linker” is a sulfur-linker selected from the group consisting of thiols, thioethers, disulfides, sulfoxides, sulfones and thiolates, and is coupled to the non-side chain carboxylic group of B, and the C═O and NH groups denoted in the formula belong to residues A and B.

The invention further relates to the use of a peptide conjugate having formula (I), in therapy.

The invention yet further relates to the use of a peptide conjugate having formula (I), for manufacturing a tumor targeting therapeutic agent.

### BRIEF DESCRIPTION OF THE FIGURES

FIG. 1: denotes the observed high tumor-to-muscle ratio that proves the highly selective binding of two peptide-effector conjugates (MJ015 and MJ053) to C8161 tumors. MJ015 in black and MJ053 in white; A=tumor, B=heart, C=lung, D=liver, E=spleen, F=small intestine and G=brain.

FIG. 2: denotes selective binding of immobilized peptide-effector conjugate (MJ013) to the colon cancer cell lines HCT-15 (A), HCT-15-LM1 (B), tongue carcinoma HSC-3 (C) and melanoma cell line C8161T (D), whereas the control cell lines, mouse fibroblast cell line NIH3T3 (E) and murine endothelial cell line SVEC4-10 (F) show significantly less binding.

FIG. 3: denotes the ratios between the Eu-content of tumors and control organs and that of muscle, after i.v. injection of the targeting agents MJ018 (black columns) and MJ069 (white columns). The tissues analysed were: A=tumor, B=heart, C=lung, D=small intestine and E=brain. The Y-axis represents the tissue-to-muscle ratios of europium content in mice.

### DETAILED DESCRIPTION OF THE INVENTION

The present invention provides a method for synthesizing improved cyclic peptides and peptide conjugates that comprise at least one functional peptide and that are easily linkable to at least one effector unit. The structure of such peptide conjugates according to the present invention closely mimics the structure of peptides that are cyclic by virtue of a cystine bridge. Thus the peptide conjugates according to the present invention retain the useful characteristics of known peptides conformationally restricted by a cystine bridge.

A highly advantageous property of cyclic peptides and peptide conjugates synthesized according to the present invention is their stability in vitro and in vivo, i.e. a long half-life. Since blood, tissues and cells contain and produce proteases and peptidases, it is important that peptide conjugates used in vivo are resistant to attacks by these enzymes. The functional peptides have to remain intact during a time period of minutes to several hours for clinical, experimental or research use. A well-known way of protecting peptides from enzymatic degradation, and of increasing their stability, is to cyclize the functional peptide of the conjugate. The present invention provides a method for easy and convenient cyclization of peptides. Further, the peptide conjugates produced by the method are considerably more stable than the conventionally used cystine-bridged peptides.

Another important feature of the present invention, the functional peptide conjugates as well as their synthesis and uses thereof, is the ease by which such peptide conjugates can be coupled or linked to different effector units in a site-specific way.

Peptide conjugates according to the present invention can be coupled e.g. to any thiol-reactive effector unit. The coupling of such a thiol-reactive effector unit can be performed either in conjunction with the peptide synthesis, or it can be done at any later time, in any standard laboratory without sophisticated synthesis facilities. The ease and similarity by which peptide conjugates can be bound to various effector units give extra value in all applications where multiple conjugates are beneficial, e.g. in therapy and in diagnosis.

The term “amino acid” is to be interpreted herein to include also diamino, triamino, oligoamino and polyamino acids and alcohols; dicarboxyl, tricarboxyl, oligocarboxyl and polycarboxylamino acids; dihydroxyl, trihydroxyl, oligohydroxyl and polyhydroxylamino alcohols; and analogous compounds comprising more than one carboxyl group or hydroxyl group and one or more amino groups. Any amino acid referred to in the present application is intended to include all isomers thereof, such as optical and geometrical isomers, if not otherwise stated.

For the purpose of the present invention, the term “functional peptide” stands for a peptide that is capable of penetrating biological membranes, organs or tissue, or selectively targeting and binding to desired materials. Preferably, such materials are biological, and more preferably they are animal tissues or cells. More preferably they are tumors, tumor stroma, tumor parenchyma and/or extra cellular matrix of tumors. Tumor targeting means that the functional peptides specifically bind to tumors when administered to a human or animal body. More specifically, the functional peptides may bind to a cell surface, to a specific molecule or structure on a cell surface or within the cells, or they may associate with the extra cellular matrix present between the cells. The functional peptides may also bind to the endothelial cells or the extra cellular matrix of tumor vasculature. The functional peptides may bind also to the tumor mass, tumor cells and extra cellular matrix of metastases. A functional peptide suitable for use in the present invention thus has a pre-determined amino acid sequence, which depends on the desired function of the peptide. The pre-determined amino acid sequence should preferably not comprise amino acids such as cysteine that have free (unbound) thiol functionality. Preferably a functional peptide has an amino acid sequence comprising 3-30 amino acids, more preferably 3-12, and still more preferably 3-9 amino acids.

Generally, the terms “targeting” or “binding” stand for adhesion, attachment, affinity or binding of the peptide conjugates of this invention to materials, biological materials, tumors, tumor cells and/or tumor tissue to the extent that the binding can be objectively measured and determined e.g., by measurement of signal from a radio- or fluorochrome labeled peptide, by competition experiments in vivo or ex vivo, on tumor biopsies in vitro or by immunological staining in situ, or by other methods known by those skilled in the art. Peptide conjugates according to the present invention are considered to be “bound” to the target in vitro, when the binding is strong enough to withstand normal sample treatment, such as washes and rinses with buffers, physiological saline or other physiologically acceptable salt or buffer solutions at physiological pH, or when bound to a target in vivo long enough for the effector to exhibit its function on the target.

The binding of the functional peptide conjugates provided by the method of the present invention is “selective” meaning that they do not bind to unintended or non-selected targets such as normal cells and organs, or bind to such to a significantly lower degree as compared to the desired or selected target.

The term “cyclic peptide” is herein used to encompass peptides that comprise at least one functional peptide and that are rendered cyclic by a lactam bridge, as described herein. Lactams can be of several types, such as “head-to-tail” (carboxy terminus to amino terminus), “head-to-side chain” and “side chain-to-head” (carboxy or amino terminus respectively to a side chain amino or carboxyl group) and “side chain-to-side chain” (amino group of one side chain and carboxyl group of another side chain). The term “peptide conjugate” is herein used to encompass conjugates that comprise a cyclic peptide according to the present invention, and at least one sulfur-linker. Such peptide conjugates may optionally comprise additional moieties, such as additional amino acids outside said cyclic peptide, optional spacers and modifying units.

The term “peptide-effector conjugate” is herein used to encompass any combinations of peptide conjugates according to the present invention and effector units. The peptide conjugate and the effector unit is coupled with a sulfur-linker according to the present invention.

It is a first object of the present invention to provide a method for synthesizing cyclic peptides comprising a “head-to-side chain” lactam, preferably formed by a D-amino acid at the N-terminus of a functional peptide and an L-amino dicarboxylic acid C-terminally located from said functional peptide, such that a sulfur-linker can be coupled to said cyclic peptide, and peptide conjugates comprising such.

A peptide according to the present invention is synthesized using conventional peptide synthesis methods known in the art, such as solid-phase methods (FMOC-, BOC-, and other protection schemes, various resin types), solution methods (FMOC, BOC and other variants) and combinations of these. Automated devices for use in peptide synthesis are commercially available, as are also routine synthesis and purification services.

Protecting groups are often used for temporary blocking of competing groups/functions such as amino, carboxyl, hydroxyl, guanyl and thiol groups, to enable directed site-specific synthetic reactions. A large variety of protecting groups are known in the art, such as the base labile FMOC for amino, and moderately acid-labile BOC for amino, t-butyl for hydroxy and carboxy, 2,2,4,6,7-pentamethyldihydrobenzofuran-5-sulfonyl (i.e. Pbf) for the side chain of arginine and acid-labile trityl for thiol groups. Peptide syntheses are facilitated by an extensive supply of commercially available building blocks including protecting reagents and compounds comprising protecting groups, thus the course of the peptide synthesis may consist of sequential deprotection and coupling steps.

It is also possible to use orthogonal protecting groups enabling site-specific modifications of desired functional groups. As used herein, the term “orthogonal protection” is intended to include semi-, quasi- and pseudoorthogonal protections that are well known to a person skilled in the art. Protecting and activating groups, substances and their uses are exemplified in the Examples and described in the references cited herein, and are commonly known in the art. Professional recommendations about the use of protecting groups, as well as about common methods of peptide synthesis, are found in e.g. technical bulletins such as: “Synthesis Notes” edited by P. White, B. Dörner and R. Steinauer in “Novabiochem 2002/3 Catalog”, published by Calbiochem-Novabiochem AG, Läufelfingen, Switzerland; or “The BACHEM Practice of SPPS” compiled by M. Mergler & J. P. Durieux, and published in 2000 by BACHEM AG Bubendorf, Switzerland.

The peptides according to the present invention may be synthesized by methods based on the use of orthogonally protected amino acids, as described in e.g., International Patent Publication WO 2004/031218, incorporated herein by reference.

In the method according to the present invention the peptide synthesis preferably starts by providing a dicarboxylic amino acid. Preferably this amino acid is an L-amino acid, such as glutamic acid or aspartic acid. Any side chain comprising a carboxyl group is orthogonally protected by e.g. acid labile protecting groups such as O-2-phenylisopropyl.

In a preferred embodiment of the present invention, a peptide according to the present invention is synthesized on a solid support, starting from the C-terminus, using conventional solid phase techniques. A suitable solid support is for example a peptide synthesis resin preloaded with any aminoalkanethiol, e.g. a cysteamine 2-chlorotrityl resin.

Other resins for solid-phase synthesis are well known in the art, and examples of them are described in the Examples and in the references cited in the present application.

In another embodiment, the peptide according to the present invention is synthesized in solution starting from the C-terminus, using conventional solution phase techniques. In solution based synthesis it is possible that optional units at the C-terminal dicarboxylic amino acid are coupled beforehand to the α-carboxyl of the dicarboxylic amino acid by using protection methods compatible with the synthesis of the peptide. The protection of the side chain carboxyl, e.g. allyl ester has to be orthogonal with respect to functional groups that are not involved in the cyclization, but the amino-terminus may be protected regularly according to the synthesis technique chosen.

In order to achieve a peptide sequence having the intended function, suitable amino acids are added to the N-terminus of said dicarboxylic amino acid, using conventional peptide synthesis techniques. Intended function include, for example specific binding ability, strong affinity for a certain target structure or molecule, and tumor targeting ability.

The synthesis of said functional peptide is performed using any conventional peptide synthesis techniques or their modifications, preferably by adding constituent amino acids, or peptide fragments, one by one to the growing peptide. However, in some embodiments it may be preferable to form said functional peptide separately and thereafter couple it to the C-terminal dicarboxylic amino acid.

The amino acid sequence of such a functional peptide is predetermined according to the desired function, and a person skilled in the art may easily select what peptide sequences to use. Functional peptides useful for targeting tumor cells and tissues, are described e.g. in international patent publication WO 2004/031218, and in the examples given below. The only prerequisite of the sequences of functional peptides according to the present invention is that they may not contain cysteines.

In a step of the peptide synthesis, a D-amino acid or a β-amino acid is added to the final N-terminus of the functional peptide. An example of a preferred D-amino acid is, e.g., D-alanine. Other preferred D-amino acids are e.g. D-ornithine, D-lysine, D-glutamic acid, D-glutamine, D-arginine, D-leucine, D-phenylalanine, D-tyrosine, D-cysteine and D-homocysteine. Examples of preferred β-amino acids are β-alanine, (S)-3-amino-2-methylpropionic acid and (S)-2-aminomethyl butyric acid.

Inserting a D-amino acid or a β-amino acid at the N-terminal end of the functional peptide makes it possible to construct a short head-to-side chain lactam bridge that mimics the structure of a cystine bridge. Such a head-to-side chain lactam gives overall structural similarity as compared to a side chain-to-side chain bridge consisting of L-amino acids. Thus, it is possible to combine the use of a stability increasing D-amino acid with a structurally preferred cyclization.

After addition of this N-terminal amino acid, any protecting group is removed from the amino group of the N-terminus and any side chain protection is removed from the dicarboxylic amino acid, and thereafter the side chain is linked by an amide bond to the N-terminus, using suitable activation techniques. Suitable activations techniques, involving carboxyl function activation and/or activation of amino groups are well known in the art. Examples of suitable activation reagents for cyclization are 7-azabenzotriazol-1-yloxytris(pyrrilidino)-phosphonium-hexafluorophosphate/di-isopropyl ethylamine (in Example 2) and di-isopropylcarbodi-imide/1-hydroxy-7-azabenzotriazol.

A head-to-side chain lactam bridge is cheaper and easier to make, compared to earlier disclosed side chain-to-side chain bridges, because less protection is needed.

The cysteine-free way of rendering the functional peptide cyclic according to the present invention provides the important advantage of the possibility of coupling desired effector units, and other optional units, in a site-specific way to targeting peptides by virtue of a sulfur containing functional group. Spacers and other desired optional units can be coupled to the N-terminal amino acid if it is for example D-cysteine or other sulfur containing amino acid.

The optional step of bridge forming reaction that links said peptide conjugate to an effector unit may be performed in at least two ways. Either as a direct linkage between a sulfur containing functional group and a sulfur-reactive site, or the bridge forming reaction can start as a nucleophilic attack from e.g. a thiol group or any other sulfur containing functional group to an electrophilic site e.g. a carbonyl, the attack is followed by a re-arrangement reaction forming e.g. an amide bond and returning, modifying or losing said sulfur containing functional group. Said sulfur-linker may be provided at any of the steps in the method, before or after synthesizing the functional peptide. Thus said sulfur-linker may be coupled to the dicarboxylic L-amino acid either before it is incorporated into the synthetic peptide or after incorporation. Furthermore, said sulfur-linker may be coupled to a spacer, either before incorporation or after.

The sulfur-linker according to the present invention is such that it forms a sulfur containing bridge when coupling said cyclic peptide to said effector unit. The sulfur-linker carries at least one sulfur atom, and may be selected from the group consisting of: A thiol group, a protected thiol group, a metal thiolate group, a disulfide group bearing for example an aryl or a pyridyl group, thioethers, disulfides, sulfoxides, sulfones and thiolates. The sulfur-linker according to the present invention can have the general formula of HN—C₁₋₁₀—X, wherein X is a thiol group, a protected thiol group, a metal thiolate group, a disulfide group bearing for example an aryl or a pyridyl group, a thioether, a disulfide, a sulfoxide, a sulfone or a thiolate. More preferably the sulfur-linker is a thiol, such as aminoalkanethiol, cysteamine, homocysteine or cysteine.

In a preferred embodiment of the present invention, where the synthesis is performed on a solid resin, the cyclic peptide is subsequently removed from the resin using conventional techniques and purified by chromatography.

In a preferred embodiment, wherein said resin is preloaded, e.g., a cysteamine 2-chlorotrityl resin, this step provides a cyclic peptide, having a sulfhydryl group at its C-terminal end, which provides a useful linking site for further coupling of additional desired effector units to the peptide.

In another embodiment of the present invention, where the peptide synthesis is performed in solution, said sulfur-linker may be added to said functional peptide prior to or after the cyclization of said functional peptide.

The synthesis steps of the present invention may also proceed by coupling together building blocks comprising two or more pre-coupled amino acids with suitable protecting groups. The building blocks to be coupled may also comprise other moieties besides amino acids, such as spacers or linkers, which are coupled or uncoupled to spacers.

Non-limiting examples of different synthesis strategies useful in a method according to the present invention are:

a) A linker containing a sulfur-bond forming site is coupled via said sulfur-bond to a solid-phase synthesis resin and a spacer is coupled to the other end of the linker. A side chain protected dicarboxylic amino acid is coupled to the spacer followed by regular solid-phase peptide synthesis starting from the dicarboxylic amino acid. After the synthesis of the functional peptide, ending in the N-terminal coupling of the desired amino acid, the side chain protecting group of the dicarboxylic amino acid is removed and the functional peptide is rendered cyclic as described above. The peptide conjugate comprising the cyclic peptide, the spacer and the linker is removed from the synthesis resin by breaking the bond at said site, which returns the sulfur-bond forming potential of said linker.

b) A side chain protected dicarboxylic amino acid is coupled to a solid-phase synthesis resin and a functional peptide is synthesized from it using regular solid-phase peptide synthesis methods. After the synthesis of the functional peptide the side chain protecting group from the dicarboxylic amino acid is removed and the functional peptide is rendered cyclic as described above. The cyclic functional peptide is removed from the solid phase by releasing the non-side chain carboxylic group, which is then linked to a spacer with a peptide bond. The spacer is further coupled to a linker containing a site capable of forming a sulfur-bond with any effector unit.

c) A solution based peptide synthesis starts from a side chain protected dicarboxylic amino acid, to which a linker, according to the present invention, has been pre-coupled. The peptide synthesis is continued from the N-terminus of the dicarboxylic amino acid, using conventional solution based peptide synthesis methods, to form a functional peptide. The peptide synthesis ends with a N-terminal D- or F-amino acid, after which the protecting groups of the non-side chain amino at the N-terminus and the side chain of the dicarboxylic amino acid is removed and the peptide is rendered cyclic. The peptide conjugates synthesized by a method according to the present invention are stable and may be stored for further use.

In one embodiment of the present invention said peptide conjugate comprising a sulfur-linker according to the present invention is coupled to an effector by said sulfur-linker to produce an end product for a desired application.

As the functional peptide does not contain any cysteines, it is possible to use units capable of forming sulfur-bonds for site-specific coupling of effector units to the peptide conjugate. In one useful embodiment of the present invention, said sulfur-linker comprises a thiol and said effector unit comprises a thiol-reactive unit. Thiol-reactive units have a thiol-reactive moiety e.g. a haloacetyl or maleimido groups, which produce a stable thioether bond (i.e. sulfide bond) with a thiol group, or e.g. a pyridyidisulfide group producing a recombined disulfide with a thiol group (by loss of pyridylthiol).

Said thiol-reactive moiety can be connected to molecules bearing NH or OH groups, for example by treatment with iodoacetic anhydride or with a bifunctional crosslinker such as an N-hydroxysuccinimid ester of 3-maleimido propionic acid or maleimidobenzoic acid or other carboxylic acids.

Site-specific coupling of an effector unit to a peptide conjugate synthesized according to the present invention may be performed at any stage as the peptide conjugates are stable and have a long shelf lifetime. Coupling of said effector unit to said peptide conjugate is not dependent on the synthesis path according to the present invention used for the peptide conjugate.

The present invention further provides peptide conjugates that retain the targeting properties of a functional peptide, and which further comprise a sulfur-linker to which an effector unit of choice may be coupled. The peptide conjugates according to the present invention comprise a functional peptide moiety, which is rendered cyclic by a head-to-side chain lactam bridge. A sulfur-linker according to the present invention is selected from the group consisting of thiols, thioethers, disulfides, sulfoxides, sulfones and thiolates.

A peptide conjugate according to the present invention may further comprise a spacer moiety between said cyclic peptide and said sulfur-linker moiety.

More specifically the present invention provides improved peptide conjugates comprising a head-to-side chain lactam bridge rendering the functional peptide cyclic, such that said cyclic peptide may be coupled to a sulfur-linker.

In a preferred embodiment of the present invention, said lactam bridge is formed by a D-amino acid at the N-terminus of a functional peptide and an L-amino dicarboxylic acid C-terminally located from the functional peptide, such that said L-amino dicarboxylic acid may be coupled via its C-terminal carboxyl to one or more moieties selected from the group consisting of a spacer moiety, a sulfur-linker and a solid phase. Preferably, said D-amino acid is D-alanine, and said dicarboxylic amino acid is selected from the group consisting of glutamic acid and aspartic acid. Other suitable D-amino acids useful in a peptide conjugate according to the present invention are D-ornithine, D-lysine, D-glutamic acid, D-glutamine, D-arginine, D-leusine, D-phenylalanine, D-tyrosine, D-cysteine and D-homocysteine.

A sulfur-linker according to the present invention carries at least one sulfur atom, and may be selected from the group consisting of: A thiol group, a protected thiol group, a metal thiolate group, a disulfide group bearing for example aryl or pyridyl group, thioethers, disulfides, sulfoxides, sulfones and thiolates. The sulfur-linker according to the present invention can have the general formula of HN—C₁₋₁₀—X, wherein X is a thiol group, a protected thiol group, a metal thiolate group, a disulfide group bearing for example an aryl or a pyridyl group, a thioether, a disulfide, a sulfoxide, a sulfone or a thiolate. More preferably the sulfur-linker is a protected or solid support bound thiol, such as aminoalkanethiol, cysteamine, homocysteine or cysteine.

A peptide conjugate according to the present invention may further comprise a spacer moiety.

Preferred spacer moieties are combinations of units having amino and/or carboxyl functionalities so that they can be included by means of regular peptide synthesis methods, especially natural and non-natural amino acids and their like.

The present invention thus relates to a cyclic peptide conjugate according to formula (I):

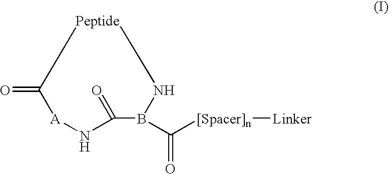

wherein

n is 0-1

“Peptide” is a functional peptide capable of penetrating biological membranes, organs or tissue or targeting and binding selectively to desired materials or tumor tissue and said peptide comprises 3-30 amino acids, except amino acids having a thiol group,

A is a residue of a D-amino acid or a β-amino acid,

B is a residue of a dicarboxylic amino acid,

“Spacer” is moiety providing distance between said sulfur-linker and said cyclic peptide and/or solubility enhancing properties to the peptide conjugate,

“Linker” is a sulfur-linker selected from the group consisting of thiols, thioethers, disulfides, sulfoxides, sulfones and thiolates, and is coupled to the non-side chain carboxylic group of B, and the C═O and NH groups denoted in formula (I) belong to residues A and B. Residue A is coupled to the N-terminus of “Peptide”, residue B is coupled to the C-terminus of “Peptide” and the side chain of residue B is coupled to the residue A.

In formula (I), “Peptide” denotes any functional peptide, preferably a tumor targeting peptide. More specifically “peptide” is a tumor targeting peptide having an amino acid sequence comprising 3-30 amino acids, preferably 3-12, more preferably 3-9, and most preferably 3-6 amino acids. “Spacer” denotes a moiety that separates the peptide part from the linker part of the cyclic peptide.

The “Spacer”, according to the present invention may further comprise additional moieties, such as solubility modifying units for modifying the solubility of conjugates; stabilizer units for stabilizing the structure of the conjugates during synthesis, modification, processing, storage or use in vivo or in vitro; charge modifying units for modifying the electrical charges of the conjugates or their starting materials; reactivity modifier units; internalizing units or enhancer units for enhancing targeting or uptake of the conjugates; adsorption enhancer units, such as fat soluble or water soluble structures that for example enhance absorption of the conjugates in vivo; or other related units.

A large number of suitable solubility modifier units are known in the art. Suitable solubility modifier units may comprise, for example:
for increasing aqueous solubility: molecules comprising SO³⁻, O—SO³⁻, COOH, COO⁻, NH₂, NH³⁺, OH, phosphate groups, guanidino or amidino groups or other ionic or ionizable groups or sugar-type structures;
for increasing fat solubility or solubility in organic solvents: units comprising (long) aliphatic branched or non-branched alkyl or alkenyl groups, cyclic non-aromatic groups such as the cyclohexyl group, aromatic rings or steroidal structures.

A large number of units known in the art can be used as stabilizer units, e.g. bulky structures (such as tert-butyl groups, naphthyl and adamantyl and related radicals etc.) for increasing steric hindrance, and D-amino acids and other unnatural amino acids (including beta-amino acids, omega-amino acids, amino acids with very large side chains etc.) for preventing or hindering enzymatic hydrolysis.

Units comprising positive, negative or both types of charges can be used as charge modifier units, as can also structures that are converted or can be converted into units with positive, negative or both types of charges.

Units that are susceptible to hydrolysis (either spontaneous chemical hydrolysis or enzymatic hydrolysis by the body's own enzymes or enzymes administered to a patient) may be very advantageous in cases where it is desired that the effector units are liberated from the conjugates e.g. for internalization, intra- or extra cellular DNA or receptor binding. Suitable units for this purpose include, for example, structures comprising one or more ester or acetal functionality. Various proteases may be used for the purposes mentioned. Many groups used for making pro-drugs are suitable for the purpose of increasing or causing hydrolysis, lytic reactions or other decomposition processes.

Examples of useful spacer moieties are: Teg, i.e. NH—(CH₂CH₂—O)₃—CH₂—CO; (Teg-E)₂, i.e. Teg-Glu-Teg-Glu, and Tegc, i.e. NH—(CH₂CH₂—O)₃—CH₂CH₂—CO.

For the purposes of this invention, the term “effector unit” denotes molecules or radicals or other chemical entities or large particles such as colloidal particles and their like; liposomes, nanoparticles or microgranules and their like. Suitable effector units may also comprise nanodevices or nanochips or their like; or a combination of any of the aforementioned, and optionally chemical structures for the attachment of the constituents of the effector unit to a conjugate according to the present invention. Effector units may also contain moieties that modify the stability or solubility or other related properties of the effector units.

Preferred effects provided by the effector units according to the present invention are therapeutic (biological, chemical or physical) effects on a targeted tumor; properties that enable the detection or imaging of tumors or tumor cells for diagnostic purposes; or binding abilities that relate to the use of the targeting agents in different applications.

A preferred (biological) activity of the effector units according to the present invention is a therapeutic effect. Examples of such therapeutic effects are cytotoxicity, cytostatic effects, antimicrobial effects, antiviral effects, ability to cause differentiation of cells or to increase their degree of differentiation or to cause phenotypic changes or metabolic changes, chemotactic activities, immunomodulating activities, pain relieving activities, radioactivity, ability to affect the cell cycle, ability to cause apoptosis, hormonal activities, enzymatic activities, ability to transfect cells, gene transferring activities, ability to mediate “knock-out” of one or more genes, ability to cause gene replacements or “knock-in”, ability to decrease, inhibit or block gene or protein expression, antiangiogenic activities, ability to collect heat or other energy from external radiation or electric or magnetic fields, ability to affect transcription, translation or replication of the cell's genetic information or external related information, or to affect post-transcriptional or post-translational events, and so on.

One preferred therapeutic application enabled by the effector units according to the present invention is the use of neutron capture therapy-active (NCT-active) substances as effector units. By NCT-active substances is meant any substance that by virtue of its ability to become radioactive by capture of slow neutrons can be used for neutron capture therapy (i.e. that emits radiation after having captured slow neutrons).

Examples of preferred functions of the effector units according to the present invention suitable for detection are radioactivity, paramagnetism, ferromagnetism, ferrimagnetism, or any type of magnetism, or ability to be detected by NMR spectroscopy, or ability to be detected by EPR (ESR) spectroscopy, or suitability for PET imaging (PET-active substances) and/or SPECT imaging (SPECT-active substances). By PET-active substances is meant any substance that can be used for positron emission tomography (PET). By SPECT-active substances is meant any substance that can be used for single photon emission computer tomography (SPECT) by virtue of its ability to emit photons.

Other examples of preferred properties of the effector units according to the present invention suitable for detection include presence of an immunogenic structure, or the presence of an antibody or antibody fragment or antibody-type structure, or the presence of a gold particle, or the presence of biotin or avidin or other protein, and/or luminescent and/or fluorescent and/or phosphorescent activity or the ability to enhance detection of tumors, tumor cells, endothelial cells or metastases in electron microscopy, light microscopy (UV and/or visible light), infrared microscopy, atomic force microscopy or tunneling microscopy, and so on.

Preferred detectable substances according to the present invention may comprise a chelator; a complexed metal such as a rare earth metal, a paramagnetic metal, a fluorescent metal (e.g. Eu, Tb or Ho), a radioactive metal, a PET-active substance or a SPECT-active substance; an enriched isotope; radioactive material such as a beta-emittor or an alpha-emittor; a paramagnetic substance; an affinity label; a fluorescent label (e.g. fluorescein or rhodamine) or a luminescent label.

Preferred binding abilities of an effector unit according to the present invention include, for example:

a) ability to bind metal ion(s) e.g. by chelation,

b) ability to bind a cytotoxic, apoptotic or metabolism affecting substance or a substance capable of being converted in situ into such a substance,

c) ability to bind to a substance or structure such as a histidine tag or other tag,

d) ability to bind to an enzyme or a modified enzyme,

e) ability to bind to biotin or analogues thereof,

f) ability to bind to avidin or analogues thereof,

g) ability to bind to integrins or other substances involved in cell adhesion, migration, or intracellular signalling,

h) ability to bind to phages,

i) ability to bind to lymphocytes or other blood cells,

j) ability to bind to any preselected material by virtue of the presence of antibodies or structures selected by biopanning or by other methods,

k) ability to bind to material used for signal production or amplification,

l) ability to bind to therapeutic substances.

Such binding may be the result of e.g. chelation, formation of covalent bonds, antibody-antigen-type affinity, ion pair or ion associate formation, specific interactions of the avidin-biotin-type, or the result of any type or mode of binding or affinity.

In a preferred embodiment according to the present invention said effector unit is a solid phase, such as a microtiter plate.

One especially useful aspect of the peptide conjugates according to the present invention is that an effector moiety may be added to bulk-produced peptide conjugates, without the need of highly specialized peptide synthesis facilities and skills.

Peptide conjugates according to the present invention show improved plasma stability and are useful in therapy, diagnosis and research.

Peptide conjugates according to the present invention are useful in a wide variety of applications. Such applications may be clinical, experimental or research undertakings of different kinds. Examples of such applications may be e.g. detection of certain biological structures in various biological or clinical samples, e.g. on tissue sections or in tissue lysates. Another important research application is the use of functional peptides in the binding, enrichment and isolation of desired biomolecules from tissue or cell lysates. In most such applications it is highly advantageous that the functional peptide is used as a stable peptide conjugate that is resistant to degradation, provides the ability to be detected or to be coupled to some form of solid support, without loosing its function.

Peptide conjugates according to the present invention have the additional advantage that desired effector units can be coupled to the conjugates using highly site-specific coupling by e.g. a thioether bond. Coupling by form ation of a thioether has many benefits, as the thioether bond is very stable and easily formed in water and in many other solvents without special activation reagents, conditions or specific equipments.

### EXAMPLES

The following examples are given to further illustrate preferred embodiments of the present invention, but are not intended to limit the scope of the invention. It will be obvious to a person skilled in the art, as technology advances, that the inventive concept can be implemented in various ways. The invention and its embodiments are thus not limited to the examples described herein, but may vary within the scope of the claims.

### Example 1

General Procedures for Peptide Synthesis: Manual Solid Phase Synthesis. Mass Spectral Measurements.

All manual synthetic procedures were carried out in a sealable glass funnel equipped with a sintered glass filter disc of porosity grade between 2 and 4, a polypropene or phenolic plastic screw cap on top (for sealing), and two PTFE key stopcocks: one beneath the filter disc (for draining) and one at sloping angle on the shoulder of the screw-capped neck (for argon gas inlet).

The funnel was loaded with the appropriate solid phase synthesis resin and solutions for each treatment, shaken effectively with the aid of a “wrist movement” bottle shaker for an appropriate period of time, followed by filtration effected with a moderate argon gas pressure.

The general procedure of one cycle of synthesis (=the addition of one amino acid unit) was as follows:

The appropriate synthesis resin loaded with approximately 0.25 mmol of FMOC-peptide (=peptide whose amino-terminal amino group was protected with the 9-fluorenylmethyloxycarbonyl group) consisting of one or more amino acid units having recommended protecting groups; approximately 0.5 g of resin (0.5 mmol/g) was treated in the way described below, each treatment step comprising shaking for one to two minutes with 10 ml of the solution or solvent indicated and filtration if not mentioned otherwise.

‘DCM’ means shaking with dichloromethane, and ‘DMF’ means shaking with N,N-dimethylformamide (DMF may be replaced by NMP, i.e., N-methylpyrrolidinone).

The steps of the treatment were:

1. DCM, shaking for 10-20 min;

2. DMF;

3.20% (by volume) piperidine in DMF for 5 min;

4.20% (by volume) piperidine in DMF for 10 min;

5. to 7. DMF;

8. to 10. DCM;

11. DMF;

12. DMF solution of 0.75 mmol of activated amino acid (preparation described below), shaking for 2 hours;

13. to 15. DMF;

16. to 18. DCM.

After the last treatment (18) argon gas was led through the resin for approximately 15 min and the resin was stored under argon (in the sealed reaction funnel if the synthesis was to continue with further units).

Activation of the 9-fluorenylmethyloxycarbonyl-N-protected amino acid (FMOC-amino acid) to be added to the amino acid or peptide chain on the resin was carried out, using the reagents listed below, in a separate vessel prior to treatment step no. 12. Thus, the FMOC-amino acid (0.75 mmol) was dissolved in approximately 3 ml of DMF, treated for 1 min with a solution of 0.75 mmol of HBTU dissolved in 1.5 ml of a 0.5 M solution of HOBt in DMF, and then immediately treated with 0.75 ml of a 1.07 ml of a 1.4 M DIPEA solution in DMF for 5 min (or half a minute in the exceptional activations described next); exceptionally 2,4,6-trimethylpyridine (TMP, also called collidine) was used instead of DIPEA in the case of the activation of FMOC-Cys(Trt)-OH and, FMOC-Ser(tBu)-OH, also the time of activation was shortened in these cases as well as in the activation of FMOC-Arg(Pbf)-OH (that was activated by usual reagents).

The activation reagents used for activation of the FMOC-amino acid were as follows:

HBTU, i.e. 2-(1H-benzotriazol-1-yl)-1,1,3,3-tetramethyluronium hexafluorophosphate, CAS No. [94790-37-1], Applied Biosystems Cat. No. 401091, molecular weight: 379.3 g/mol;

HOBt, i.e. 1-Hydroxybenzotriazole, CAS No. 2592-95-2, molecular weight 135.12 g/mol, Acros Organics Cat. No. 169161000; DIPEA, i.e. N,N-Diisopropylethylamine, CAS No. 7087-68-5, molecular weight 129.24 g/mol, Acros Organics Cat. No. 115221000.

The procedure described above is repeated in several cycles using different FMOC-amino acids, containing suitable protecting groups, to produce a “resin-bound” peptide (i.e., resinous source of an appropriate peptide). The procedure provides also a way to connect certain spacer units, for instance Teg i.e. 11-amino-3,6,9-undecanoyl moiety (by the reagent FMOC-Teg-OH), to the resin-bound peptide. Also the very first unit (at the C-terminal end of the sequence) can be connected to resin e.g. cysteamine resin by means of this general coupling method described above; in the case of cysteamine resin the initial treatment with piperidine (steps 3 to 11) is not necessary at the first cycle. The first cycle was carried out with 0.25 mmol of activated reagent in step 12 above (instead of usual 0.75 mmol) followed by resin capping between steps 12 and 13 by means of acetylation for 30 min using reagent mixture: 2 ml of acetic anhydride and 1 ml of 2,4,6-trimethylpyridine mixed in 4 ml of DMF.

After lactam bridge formation according to the process described below in Example 2, the resin was washed with DCM, dried at argon flow and treated with three portions of the cleavage reagent mixture described below (each about 10 ml), each for one hour. The treatments were carried out under argon atmosphere in the way described above. After three hours from the beginning of the treatment the TFA solutions obtained by filtration were concentrated under reduced pressure using a rotary evaporator and were recharged with argon.

Cleavage from the resin was carried out using the following reagent mixture:

trifluoroacetic acid (TFA) 92.5 vol-%, water 5.0 vol-%, and ethanedithiol 2.5 vol-%.

The cleavage mixture described above also simultaneously removed the following protecting groups: Tert-butoxycarbonyl (Boc) as used for protection of side chain amino group; tert-butyl ester (OtBu)/(tBu) as used for protection of side chain carboxyl group of glutamic acid or of hydroxyl group of tyrosine, and trityl (Trt) as used for protection of side chain amide or thiol.

Purification was performed by reversed phase high-performance liquid chromatographic (HPLC) method using a “Waters 600” pump apparatus with a C-18 type column of particle size 10 micrometers, and a linear eluent gradient whose composition was changed during 30 minutes from 99.9% water/0.1% TFA to 99.9% acetonitrile/0.1% TFA; in some instances (indicated below) the eluent was buffered by 0.05 M ammonium acetate instead of 0.1% TFA. The dimensions of the HPLC columns were 25 cm×21.2 mm (Supelco cat. no. 567212-U) and 15 cm×10 mm (Supelco cat. no. 567208-U). Detection was based on absorbance at 218 nm and was carried out using a “Waters 2487” instrument. The fraction indicated by right mass spectrum was collected as product and lyophilized; to enhance the purification eluent composition limits were adjusted to achieve applicable gradient.

The compound synthesized this way is constructed from “right to left” in the conventionally (also in this text) presented sequence, i.e. starting from the C-terminal end of the peptide chain.

Mass spectral method employed: Matrix Assisted Laser Desorption Ionization−Time of Flight (MALDI−TOF).

Type of the instrument: Bruker Ultraflex MALDI TOF/TOF mass spectrometer.

Supplier of the instrument: Bruker Daltonik GmbH, Bremen, Germany.

MALDI-TOF Positive Ion Reflector Mode:

External standards: Angiotensin II, angiotensin II, substance P, bombesin, ACTH(1-17) ACTH(18-39), somatostatin 28 and bradykinin 1-7 Matrix: alpha-cyano-4-hydroxycinnamic acid (2 mg/ml solution in aqueous 60% acetonitrile containing 0.1% of trifluoroacetic acid, or acetone only for acid sensitive samples).

MALDI-TOF Negative Ion Reflector Mode:

External standards: cholecystokinin and glucagon or [Glu1]-fibrinogen peptide B.

Matrix: alpha-cyano-4-hydroxycinnamic acid (saturated solution in acetone).

Sample preparation: The specimen was mixed at a 10-100 picomol/microliter concentration with the matrix solution as described and dried onto the target.

Ionization by “shooting” in vacuo by nitrogen laser at wavelength 337 nm. The Voltage of the probe plate was 19 kV in positive ion reflector mode and −19 kV in the negative ion reflector mode.

General Remarks about the Spectra (Concerning Positive Ion Mode Only):

In all cases the M+1 (i.e., the one proton adduct) signal with its typical fine structure based on isotope satellites was clearly predominant. In almost all cases, the M+1 signal pattern was accompanied by a similar but markedly weaker band of peaks at M+23 (Na⁺ adduct) and at M+39 (K⁺ ad-duct).

The molecular mass values reported within synthesis examples correspond to the most abundant isotopes of each element, i.e., the ‘exact masses’.

### Example 2

### General Procedure for the on-Resin Formation of a Head-to-Side-Chain Lactam Bridge between D-Alanine and Glutamic Acid

A peptide was synthesized according to the general procedure described above in Example 1 starting from the C-terminus. After any needed units (outside the C-terminus of the prospective cyclic peptide) were coupled to the peptide synthesis resin, an orthogonally protected glutamic acid: γ-2-phenylisopropylester of α-N-Fmoc-glutamic acid [i.e. FMOC-Glu(2-O-Ph-i-Pr)—OH, Novabiochem Cat. No. 04-12-1199, Molecular Weight 487.5 g/mol] was coupled to the resin prior to the units of the functional peptide. Finally FMOC-D-alanine (FMOC-D-Ala-OH, CAS No. 79990-15-1, Novabiochem Cat. No. 04-13-1006, Molecular Weight 311.3 g/mol) was coupled to the N-terminal end of the functional peptide.

To produce a lactam bridge between the N-terminus of D-alanine and the side chain of γ-2-phenylisopropylester protected glutamic acid the procedure for one cycle as described in Example 1 was carried out with the exception that an additional deprotection treatment (removal of the O-2-Ph-i-Pr protection of the side chain of Glu) of the resin was carried out after step 10 by shaking, during a total of 10 min, with four portions of 2% TFA (0.2 ml) solution in DCM (9.7 ml) containing 1% of tri-isopropylsilane (0.1 ml), each for two minutes followed by rapid filtration. Then the resin was washed with 0.2 M DIPEA in DMF followed by washing steps 8-11. Next the resin was shaken for 5 hours with 0.75 mmol of the activation reagent PyAOP (i.e. 7-azabenzotriazol-1-yloxytris-(pyrrolidino)-phosphonium-hexafluorophosphate, Applied Biosystems, CAS No. 156311-83-0, Cat. No. GEN076533, molecular weight 521.4 g/mol) in 5 ml of DMF containing 1.5 mmol of DIPEA followed by final washings according to steps 13-18. Therefore this coupling was intramolecular without any FMOC-amino acid added in step 12. Then the resin was washed with DMF and DCM before the cleavage process described in Example 1.

### Example 3

### Synthesis of Sulfhydryl-Bearing Cyclic Peptide Conjugates

The synthesis of lactam bridged conjugates a*-peptide-E*-spacer-EAT i.e. D-Ala*-peptide-Glu*-spacer-NH—CH₂CH₂—SH (the asterisk is for indicating the presence of a lactam bridge between N-terminus of D-Ala* and side chain COOH group of Glu*) comprising a cyclic peptide a*-peptide-E* and sulfhydryl bearing linker via optional spacer units at the C-terminus of the targeting unit was carried out manually according to the general protocol described in Example 1 and was based on cysteamine-2-chlorotrityl resin and solid phase FMOC-chemistry and on use of regular protected amino acid reagents, and building blocks that may be synthetically used like amino acids, e.g. FMOC-Teg-OH or FMOC-Tegc-OH included in ‘spacer’ [‘Teg’ denotes NH—CH₂CH₂—O—CH₂CH₂—O—CH₂CH₂—O—CH₂—C(O), and ‘Tegc’ denotes NH—CH₂CH₂—O—CH₂CH₂—O—CH₂CH₂—O—CH₂CH₂—C(O)]. The side chains of the amino acid reagents were protected regularly: tert-butyl for side chain oxygen (e.g. in Ser, Tyr and Glu), tBoc for tryptophan and Pbf for arginine.

The lactam bridge was prepared according to the general protocol described in Example 2 and the peptide was purified by RP-HPLC. The identification of the product was based on MALDI-TOF mass spectrum.

The major reagents in these syntheses were from Applied Biosystems, Warrington, United Kingdom or from Novabiochem, Laufelfingen, Switzerland. The spacer unit reagent FMOC-11-amino-3,6,9-undecanoic acid (for ‘Teg’) was purchased from University of Kuopio, Finland, and had been prepared as described previously (Boumrah, Deradji et al., Tetrahedron, 1997, 56: 6977-6992). The spacer unit reagent FMOC-12-amino-4,7,10-trioxadodecanoic acid (for ‘Tegc’) was purchased from NeoMPS Strasbourg, France.

The linker unit, 2-aminoethanethiol, was preloaded in the synthesis resin (Cysteamine-2-chlorotrityl Resin, Novabiochem 01-64-0107) and thus produced via the cleavage of the resin.

The following sulfhydryl-bearing cyclic peptide conjugates were prepared:

a*LRSGRGE*-Teg-EAT (Compound HP235) comprising functional peptide Leu-Arg-Ser-Gly-Arg-Gly and spacer Teg. Observed positive ion M+1: 1075.52 Da. M+Na: 1097.51 Da. Calculated isotopic M: 1074.56 Da. This synthesis was performed manually and also using an automated synthesis instrument Advanced Chem Tech 396DC.

a*SGRLGRE*-Teg-EAT (Compound HP236) comprising functional peptide Ser-Gly-Arg-Leu-Gly-Arg and spacer Teg. Observed positive ion M+1: 1075.55 Da. Calculated isotopic M: 1074.56 Da.

a*YGFVWGEE*-(Teg-E)₂-EAT (Compound MJ013) comprising functional peptide Tyr-Gly-Phe-Val-Trp-Gly-Glu and water-solubility enhancing spacer (Teg-E)₂. Observed positive ion M+Na: 1756.73 Da; M+K: 1772.71 Da. Calculated isotopic M: 1733.75 Da.

a*LRSE*-EAT (Compound KK19) comprising functional peptide Leu-Arg-Ser and directly coupled sulfhydryl bearing linker (no spacer added). Observed positive ion M+1: 616.24 Da. Calculated isotopic M: 615.32 Da.

a*LRSE*-Tegc-EAT (Compound KK18) comprising functional peptide Leu-Arg-Ser and spacer Tegc. Observed positive ion M+1: 819.38 Da. Calculated isotopic M: 818.43 Da.

a*LRSGRGE*-Tegc-EAT (Compound MJ074) comprising functional peptide Leu-Arg-Ser-Gly-Arg-Gly and spacer Tegc. Observed positive ion M+1: 1089.54 Da. Calculated isotopic M: 1088.576 Da.

a*IREE*-Tegc-EAT (Compound KK20) comprising functional peptide iLe-Arg-Glu and spacer Tegc. Observed positive ion M+1: 861.38 Da. Calculated isotopic M: 860.44 Da.

### Example 4

### Synthesis of Europium-Labelled Cyclic Peptide Conjugate

The synthesis of lactam-bridged conjugates a*-peptide-E*-spacerAEM-AABz-DTPA-Eu, i.e. D-Ala*-peptide-Glu*-spacer-NH—CH₂CH₂—S—CH₂—C(O)-p-aminobenzyl-DTPA-Eu (lactam bridge between N-terminus of a* and the side chain COOH group of E*) comprising cyclic peptide a*-peptide-E* and europium bearing DTPA chelate coupled via a thioether bond (included in ‘AEM-AABz’ linkage) and spacer units at the C-terminus of the targeting unit, was carried out in aq. NaHCO₃ at pH 8.5. The appropriate sulfhydryl-bearing peptide conjugate (a*-peptide-E*-spacer-EAT from example 3) was dissolved in 0.05 M NaHCO₃ and the Eu³⁺-chelate of 1-[4-(−2-iodoacetamido)benzyl]diethylenetriamine-N,N,N′,N″,N″-pentaacetic acid, as described below, (2-3 mol equivalent) in 0.05 M NaHCO₃ was added to the peptide solution. After pH was adjusted to 8.5, the solution was protected from light and allowed to stay overnight at 37° C. The DTPA-Eu labeled peptide was purified by RP-HPLC at water-acetonitrile eluent gradient buffered by 0.05 M ammonium acetate.

The identification of the product was based on MALDI-TOF mass spectrum.

Synthesis of 1-[4-(−2-iodoacetamido)benzyl]diethylenetriamine-N,N,N′,N″,N″-pentaacetic acid, i.e. I-AABz-DTPA (also called DTPA-IAA), and its Eu³⁺-chelate of was performed in three steps. The synthesis was started from (t-BuO)₅DTPA-Bz-NH₂ purchased from Macrocyclics, Dallas, Tex., USA (0.300 mmol), which was allowed to react with iodoacetic anhydride (0.330 mmol) and triethylamine (0.315 mmol) in dichloromethane (6 ml) at room temperature for 2 hours. The crude product was purified by Flash chromatography in silica gel column eluted by 30% ethyl acetate in hexane. Then the Flash-purified t-BuO-protected I-AABz-DTPA was deprotected over night by excess of neat TFA at room temperature and, after concentration, labelled with europium (2 mol equivalent of aqueous EuCl₃) in 0.05 M ammonium acetate buffer (3 mol equivalent) at room temperature over night. This final I-AABz-DTPA-Eu compound was purified by RP-HPLC at water-acetonitrile eluent gradient buffered by 0.05 M ammonium acetate and the desired product was identified by positive ion MALDI-TOF mass spectrometry (calculated M 816.00 Da; obtained M+H 816.96 Da).

The following specific europium-labelled peptide conjugates were prepared:

a*LRSGRGE*-Teg-AEM-AABz-DTPA-Eu (Compound MJ015) comprising functional peptide Leu-Arg-Ser-Gly-Arg-Gly and spacer Teg, i.e. NH—(CH₂CH₂—O)₃—CH₂—CO. Observed negative ion M−1: 1761.60 Da. Calculated isotopic M: 1762.65 Da.

a*SGRLGRE*-Teg-AEM-AABz-DTPA-Eu (Compound MJ022) comprising functional peptide Ser-Gly-Arg-Leu-Gly-Arg and spacer Teg. Observed positive ion M+1: 1763.59 Da, M+Na: 1785.28 Da, M+K: 1801.55 Da. Calculated isotopic M: 1762.649 Da.

a*YGFVWGEE*-(Teg-E)₂-AEM-AABz-DTPA-Eu (Compound MJ018) comprising functional peptide Tyr-Gly-Phe-Val-Trp-Gly-Glu and water-solubility enhancing spacer (Teg-E)₂ i.e. Teg-Glu-Teg-Glu. Observed negative ion M−1: 2420.67 Da. Calculated isotopic M: 2421.84 Da.

a*LRSE*-Tegc-AEM-AABz-DTPA-Eu (Compound MJ054) comprising functional peptide Leu-Arg-Ser and spacer Tegc, i.e., NH—(CH₂CH₂—O)₃—CH₂CH₂—CO. Observed positive ion M+1: 1768.60 Da, M+Na: 1790.58 Da, M+K: 1806.55 Da. Calculated isotopic M: 1767.652 Da.

a*IREE*-Tegc-AEM-AABz-DTPA-Eu (Compound MJ053) comprising functional peptide iLe-Arg-Glu and spacer Tegc. Observed positive ion M+1: 1549.56 Da, M+Na: 1571.55 Da, M+K: 1587.53 Da. Calculated isotopic M: 1548.531 Da.

### Example 5

### Synthesis of Gadolinium-Labeled Cyclic Peptide Conjugates

The synthesis was carried out by a method similar to the synthesis of europium-labeled cyclic peptide conjugates described in Example 4, but gadolinium replaced europium in this example.

The following gadolinium-labeled cyclic peptide conjugates were prepared:

a*LRSGRGE*-Teg-AEM-AABz-DTPA-Gd (compound MJ059) comprising functional peptide Leu-Arg-Ser-Gly-Arg-Gly and spacer Tegc i.e. NH—(CH₂CH₂—O)₃—CH₂CH₂—CO. Observed positive ion M+1: 1512.56 Da, M+Na: 1534.55 Da, M+K: 1550.52. Da. Calculated isotopic M: 1511.524 Da.

a*LRSE*-Tegc-AEM-AABz-DTPA-Gd (Compound MJ060) comprising functional peptide Leu-Arg-Ser and spacer Tegc. Observed positive ion M+1: 1512.56 Da, M+Na: 1534.55 Da, M+K: 1550.52 Da. Calculated isotopic M: 1511.524 Da.

a*IREE*-Tegc-AEM-AABz-DTPA-Gd (Compound MJ061) comprising functional peptide Ile-Arg-Glu and spacer Tegc. Observed positive ion M+1: 1554.49 Da, M+Na: 1576.48, M+K: 1592.44. Calculated isotopic M: 1553.534 Da.

### Example 6

### Synthesis of a Cyclic Peptide Conjugate Bearing DTPA Chelator

This example describes the synthesis of lactam bridged peptide conjugates comprising a spacer and a metal chelating linker. Such conjugates may be stored and later linked to a label or therapeutic agent of choice.

The synthesis was carried out by a method similar to the synthesis of europium-labeled cyclic peptide described in Example 4. The difference was that 1-[4-(−2-iodoacetamido)benzyl]diethylenetriamine-N,N,N′,N″,N″-pentaacetic acid i.e. I-AABz-DTPA was used as such without labelling with europlum.

The following cyclic peptide conjugate bearing DTPA chelator was prepared:

a*LRSGRGE*-Tegc-AEM-AABz-DTPA (Compound MJ082) comprising functional peptide Leu-Arg-Ser-Gly-Arg-Gly and spacer Tegc i.e. NH—(CH₂CH₂—O)₃—CH₂CH₂—CO.

Observed positive ion M+1: 1627.72 Da. Calculated isotopic M: 1626.77 Da.

### Example 7

### Synthesis of Fluorescein-Labelled Cyclic Peptide Conjugate

The synthesis of lactam bridged peptide conjugate a*-peptide-E*-spacer-AEM-Suc-Flrc, i.e., D-Ala*-peptide-Glu*-spacer-NH—CH₂CH₂—S-Suc-Flrc (lactam bridge between N-terminus of a* and the side chain COOH group of E*) comprising cyclic peptide a*-peptide-E* and fluorescein-5-succinimide coupled via a thioether bond and spacer units at the C-terminus of the targeting unit, was carried out in aqueous buffer solution at pH 7. The appropriate sulfhydryl-bearing peptide conjugate (a*-peptide-E*-spacer-EAT from example 3) and threefold excess of fluorescein-5-maleimide (Catalogue No. P4361, Promega Corporation, Madison, Wis., USA) were dissolved in 5 mM NaHCO₃, and allowed to stay 15 min at room temperature prior to stopping with 0.142 M 2-mercaptoethanol. The product was isolated on RP-HPLC by water-acetonitrile gradient buffered by 0.05 M ammonium acetate.

Particular example of fluorescein-labeled cyclic peptide conjugate:

a*LRSGRGE*-Teg-AEM-Suc-Flrc (Compound KP008) comprising functional peptide Leu-Arg-Ser-Gly-Arg-Gly and spacer Teg i.e. NH—(CH₂CH₂-0)₃—CH₂—CO. Observed positive ion M+1: 1502.54 Da. Calculated MW: 1502.9 Da.

### Example 8

### Synthesis of Indium-Labelled Cyclic Peptide Conjugate

The synthesis was carried out by a method similar to the synthesis of europium-labelled cyclic peptide conjugate described in Example 4, but indium replaced europium in this example.

The following indium-labelled cyclic peptide conjugates were prepared:

a*LRSGRGE*-Tegc-AEM-AABz-DTPA-In (compound MJ143) comprising functional peptide Leu-Arg-Ser-Gly-Arg-Gly and spacer Tegc i.e. NH—(CH₂CH₂—O)₃—CH₂CH₂—CO. Observed positive ion M+1: 1739.63 Da, M+Na: 1761.61 Da, M+K: 1777.58 Da. Calculated isotopic M: 1738.65 Da.

a*YGFVWGEE*-(Tegc-E)₂-AEM-AABz-DTPA-In (Compound MJ146) comprising functional peptide Tyr-Gly-Phe-Val-Trp-Gly-Glu and water-solubility enhancing spacer (Tegc-E)₂ i.e. Tegc-Glu-Tegc-Glu. Observed positive ion M+1: 2412.69 Da, M+Na: 2434.67 Da, M+K: 2450.64 Da. Calculated isotopic M: 2411.85 Da.

### Example 9

### Targeting Peptide Biodistribution

This example shows the biodistribution of two peptide conjugates synthesized in Example 4, a*LRSGRGE*-Teg-AEM-AABz-DTPA-Eu (Compound MJ015) and a*IREE*-Tegc-AEM-AABz-DTPA-Eu (Compound MJ053), for primary tumors of C8161 human melanoma cell line. It is shown that the tested conjugates according to the present invention selectively target to primary tumors in vivo but not to normal tissues or organs.

For production of experimental tumors 1×106 cells of C8161 line (described in Example 9) were injected subcutaneously into the flank of athymic nude mice strain (Harlan Laboratories). Tumors were harvested when they had reached a weight of about 0.1 g. Tumor-bearing mice were anesthetized by 0.02 ml/g body weight of Avertin [10 g 2,2,2-tribromoethanol (Fluka) in 10 ml 2-methyl-2-butanol (Sigma Aldrich)] intraperitoneally (i.p.).

To determine the biodistribution pattern of the conjugates, 4 nmol of MJ015 or MJ053 was injected into the tail vein of athymic nude mice in a volume of 100 μl in physiological saline solution (Baxter). The conjugate was allowed to circulate for 20 min. Mice were then perfused through the heart with 60 ml of physiological saline. Organs and tissues, including tumors were collected and weighed.

For determination of the Eu content of the tumors and control organs, 0.1-0.2 g of tissue was transferred into lysis buffer (20 mM Tris-HCl, 150 mM NaCl, 1% Nonident P40, pH 7.5) and homogenized. Eu-content was analyzed from tissue lysates prepared into the DELFIA Inducer solution (Perkin Elmer Wallac Ltd., Turku, Finland) and transferred to DELFIA Micro titration strip plates (Perkin Elmer Wallac Ltd, Turku, Finland). Time-resolved fluorescence was measured after incubation with a Wallac 1420 VICTOR3™ V plate reader using a D615 nm filter. The comparison of the amount of europium detected in the mouse tissues showed that both the conjugates MJ015 and MJ053 accumulated strongly and selectively in C8161 tumors compared to normal tissue, except for the kidney showing high signal due to excretion of the agent via these routes.

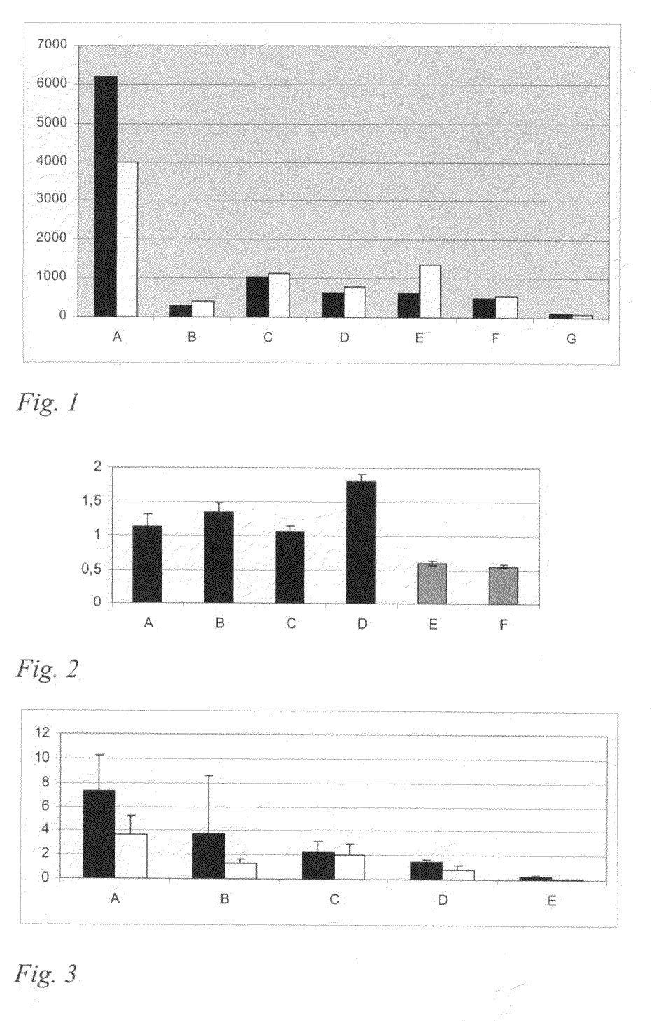

The observed high tumor-to-muscle ratio further proves the highly selective binding of MJ015 and MJ053 to C8161 tumors shown in FIG. 1. MJ015 in black and MJ053 in white; A=tumor, B=heart, C=lung, D=liver, E=spleen, F=small intestine and G=brain.

The ratio of tumor Eu-content to muscle Eu-content of conjugate MJ015 was 7.1 and of conjugate MJ053 was 3.9.

This example shows that the peptide conjugates according to the present invention have retained their highly selective tumor and metastase targeting properties after cyclization and labeling.

### Example 10

### Cell Binding Assay of Immobilized Lactam Bridged Peptide Conjugate

In this example the following cell lines and culture conditions were used:

The human colorectal cancer HCT-15 cell line (ATCC: CCL-225), called herein also “HCT-15”, was cultured in RPMI 1640 medium with 2 mM L-glutamine adjusted to contain 1.5 g/L sodium bicarbonate, 4.5 g/L glucose, 10 mM HEPES, and 1.0 mM sodium pyruvate, 1% penicillin/streptomycin, 10% fetal bovine serum.

The colorectal cancer cell line HCT-15-LM1 was developed as follows. The cell culture was started with cancer cells from lung metastases which had developed after injection of human colorectal cancer HCT-15 cells into the bloodstream of a mouse. The inoculation procedure was then repeated with HCT-15-LM1 cells leading to the establishment of another metastatic cell line, HCT-15-LM2.

The human oral squamous cell carcinoma line HSC-3, called herein “HSC-3” (JCRB Cell Bank 0623, National Institute of Health Sciences, Japan) was cultivated in 1:1 DMEM and Ham's F 12 medium containing 10% FBS 1% penicillin/streptomycin, L-glutamate and sodium pyruvate and 0.4 ng/ml hydrocortisone.

The human melanoma cell line C8161 has been described previously by Welch et al. in Int. J. Cancer, 1991, 47: 227-237. A more metastatic cell line C8161T, called herein also “C8161T”, was developed by culturing cells from a subcutaneous melanoma tumor developed after inoculation of C8161 melanoma cells on the flank of a nude mouse. The cell line was cultured in DMEM medium adjusted to contain 2 mM L-glutamine, 1% penicillin/streptomycin, and 10% fetal bovine serum.

The mouse fibroblast line NIH3T3, called herein also “NIH3T3”, has been described previously by Koga et al. in Gann, 1979, 70: 585-591. The cell line was cultured in DMEM medium adjusted to contain 2 mM L-glutamine, 1% penicillin/streptomycin, and 10% fetal bovine serum.

The mouse vascular endothelial cell line SVEC4-10, called herein also “SVEC4-10”, has been described previously by O'Connell et al. in J. Immunol., 1990, 144: 521-525. The cell line was cultured in DMEM medium adjusted to contain 2 mM L-glutamine, 1% penicillin/streptomycin, and 10% fetal bovine serum.

Preparation of plates for the assays. Wells of Reacti-Bind Maleimide activated clear strip plate (Pierce, Prod#. 15150) were coated with a peptide conjugate a*YGFVWGEE*-(Teg-E)₂-EAT, Compound MJ013 prepared in Example 3, at a concentration of 30 μg/ml. The incubation was carried out overnight at 20° C. The binding buffer containing unbound peptide was re-moved from the wells.

The wells were blocked with blocking buffer (0.5% BSA, 0.05% Tween20 in phosphate buffer saline (PBS). PBS was prepared as follows: 40 g of NaCl, 1 g of KCl, 7 g of Na₂HPO₄×2H₂O and 1 g of KH₂PO₄ were dissolved in 1000 ml of deionized H₂O). Blank wells as controls were prepared by treating empty wells with blocking buffer. The plates were incubated 1 hour 30 min at 20° C. After incubation the plate was washed three times with PBS, pH 7.4.

Cell binding assay: 75000 cells in volume of 150 μl of medium were added into coated wells and were incubated for 30 min at 37° C. After cell binding, the wells were washed with PBS for 30 min. Detection of peptide conjugate bound cells were based on the MTT (Thiazolyl blue, Sigma M-5655) tetrazolium salt assay. MTT is cleaved to water-insoluble formazan dye by the “succinate-tetrazolium reductase” system, which is active only in viable (unbound) cells. After formazan was solubilized by 10% SDS-0,01 M HCl, it was quantified with an ELISA spectrometer (ThermoLabsystems Multiscan EX) at 560 nm. 10 μl of MTT reagent and 90 μl of medium were added to the wells. The plate was incubated for three hours at 37° C. After the incubation, 100 μl of lysis buffer was added to the wells and let to incubated o/n 37° C. The following day the absorbance of plate was measured at 560 nm with an ELISA-reader (ThermoLab-systems, Multiskan EX).

The results of the cell-binding assay showing the selective binding of cancer cell lines to the targeting agents are shown in FIG. 2. The colon cancer cell lines HCT-15 (A), HCT-15-LM1 (B), tongue carcinoma HSC-3 (C) and melanoma cell line C8161T (D), bind selectively to the immobilized peptide conjugate MJ013 (FIG. 2), whereas the control cell lines, mouse fibroblast cell line NIH3T3 (E) and murine endothelial cell line SVEC₄₋₁₀ (F) show significantly less binding. The results are shown as measured absorbance at 560 nm.

### Example 11

### Plasma Stability of the Peptide Conjugates

The plasma stability was tested for two peptide conjugates according to the present invention (MJ018, MJ059), and compared to functional peptides (KK12, KK51).

Compound KK51, a lactam-bridged peptide, D-Ala*-Leu-Arg-SerGly-Arg-Gly-Glu*-NH(CH₂CH₂O)₂CH₂CH₂NH₂, comprising a lactam bridge between N-terminus of D-Ala* and side chain COOH group of Glu*, a functional peptide Leu-Arg-Ser-Gly-Arg-Gly and spacer unit NH(CH₂CH₂O)₂CH₂CH₂NH₂ at the C-terminus of the functional peptide, was synthesized manually according to the general protocol described in Example 1, based on O-Bis(aminoethyl)-ethylene glycol trityl resin (Novabiochem 01-64-0285) and solid phase FMOC-chemistry and regular protected amino acid reagents. The lactam bridge was prepared according to the general protocol described in Example 2 and the peptide was purified by RP-HPLC. The identification of the product was based on MALDI-TOF mass spectrum.). Observed positive ion M+1: 957.43 Da. Calculated isotopic M: 956.55 Da.

Compound MJ059, a*LRSGRGE*-conjugate with gadolinium chelate was synthesized as described in Example 5.

Compound MJ018, a*YGFVWGEE*-conjugate with europium chelate was synthesized as described in Example 4.

Compound KK12, a cystine-bridged peptide, Gly-Cys*-Leu-Arg-SerGly-Arg-Gly-Cys*-Gly-NH(CH₂CH₂O)₂CH₂CH₂NH₂, cyclic by virtue of a cystine bridge between the two cysteines (Cys*), and comprising a functional peptide Leu-Arg-Ser-Gly-Arg-Gly, was synthesized manually according to the general protocol described in Example 1, based on O-Bis-(aminoethyl)-ethylene glycol trityl resin (Novabiochem 01-64-0285) and solid phase FMOC-chemistry and regular protected amino acid reagents including FMOC-Cys(Trt)-OH. After FMOC-removal treatment described above (steps 1-11 in Example 1) the compound was cleaved off the resin, as described above, and cyclized in 0.05 M ammonium bicarbonate solution exposed by air at room temperature over night, and then purified by RP-HPLC. The identification of the product was based on MALDI-TOF mass spectrum. Observed positive ion M+1: 1093.51 Da. Calculated isotopic M: 1092.53 Da.

The peptide conjugates (10-20 μg) was added to a human blood plasma/PBS mixture (1:1) in a total volume of 500 μl and incubated at 37° C. After incubation 100 μl per time point (0 and 240 min) was transferred to YM-10 (Microcon, cut-off 10000 MW; manufacturer Millipore), centrifuged (10000 RPM) 15 min at 4° C. 50 μl of the spin-through material was analyzed by HPLC.

HPLC: Waters 626 pump, 600S controller, Waters 996 photo diode array detector. Column: C18, 300 Å, 5 μm, 2.1×250 mm. Eluents for the non-conjugated functional peptides: A 0.1% TFA, B 0.075% TFA/100% ACN. Gradient: 0-100% B in 60 min. Flow: 180 μl/min. Loop: 50 μl.

Eluents for the functional peptide conjugates: Eluent A: 0.05 M ammonium acetate; B: 50% 0.05 M ammonium acetate/50% ACN. Gradient: 0-100% B in 60 min. Flow: 180 μl/min. Loop: 50 μl. One-minute fractions were collected and analyzed with MALDI-TOF.

Peptide mass: Bruker Ultraflex MALDI-TOF, N₂-laser 337 nm, matrix saturated solution of α-cyano-4-hydroxycinnamic acid, reflector mode.

TABLE 1
Observed compound peak heights prior to incubation
and after four hours

| Conjugate | 0 h | 4 h |
|---|---|---|
| KK51 (lactam) | 100% | 92% |
| KK12 (cystine) | 100% | 25% |
| MJ059 (Gd/lactam) | 100% | 100% |
| MJ018 (Eu/lactam) | 100% | 100% |

This example shows that a peptide according to the present invention, cyclized via a lactam bridge, is significantly more stable than the disulfide bridged peptide. Practically no degradation of KK51 was seen after 4 h incubation in plasma. Also, the peptide conjugates according to the present invention (MJ059, MJ018) showed no degradation during the 4 h incubation in human plasma.

### Example 12

### Biodistribution of Targeting Peptide Conjugates: Comparison of Cystine- and Lactam Bridged Conjugates

The biodistribution of the targeting agents MJ018 (lactam-bridged; described in Example 4) and MJ069 (cystine-bridged; described below) comprising functional peptide YGFVWGE was compared in mice carrying primary tumors of HTC-15, a human colorectal cancer cell line.

The peptide conjugate MJ069 was prepared as follows:

N- and C-terminally modified peptide, acetyl-Cys*-Tyr-Gly-Phe-ValTrp-Gly-Glu-Cys*-Tegc-Glu-Tegc-Glu-NHCH₂CH₂NH₂, cyclic by virtue of a cystine bridge between the two cysteines (Cys*), was synthesized manually according to the general protocol described in Example 1, based on 1,2-aminoethane trityl resin (Novabiochem 01-64-0081) and solid phase Fmoc-chemistry and regular protected amino acid reagents (that are mentioned in Examples 1 and 3) including Fmoc-Cys(Trt)-OH and Fmoc-12-amino-4,7,10-trioxadodecanoic acid (i.e. Fmoc-Tegc-OH). After Fmoc-removal treatment described above (steps 1-11 in Example 1), and capping by means of acetylation for 30 min using reagent mixture: 2 ml of acetic anhydride and 1 ml of 2,4,6-trimethylpyridine mixed in 4 ml of DMF, the resin was washed with DCM, dried at argon flow and the compound was cleaved off the resin, as described above in Example 1.

The peptide was cyclized in 0.05 M ammonium acetate solution exposed by air at room temperature over night, and then purified by RP-HPLC. The identification of the peptide was based on MALDI-TOF mass spectrum. Observed positive ion M+1: 1809.74 Da. Calculated isotopic M: 1808.8 Da.

The cystine-bridged (cyclized) peptide, having C-terminal amine functionality, was labelled with europium via DTPA chelator as follows:

4.9 mg/mL (2.7 mmol/L) of the cystine-bridged peptide was stirred with 2.5 mol equivalent (6.75 mmol/L) of ITC-DTPA, i.e. 2-(4-Isothiocyanatobenzyl)-diethylenetriaminepentaacetic acid (purchased from Macrocyclics, Dallas Tex., USA), in 0.05 M aqueous NaHCO₃ at pH 9 at ambient temperature over night. Then 3 mol equivalent (8.1 mmol/L) of EuCl₃×6H₂O was added and stirring was continued for 4 h. The DTPA-europium labelled product was purified by RP-HPLC at water-acetonitrile eluent gradient buffered by 0.05 M ammonium acetate. The identification of MJ069 was based on MALDI-TOF mass spectrum. Observed positive ion M+1: 2497.80 Da. Calculated isotopic M: 2472.85 Da.

For production of experimental tumors, 22.5 million (for MJ018), and 1 million (for MJ069) HCT-15 cells were injected subcutaneously into both flanks of athymic-nu nude mice (Harlan Laboratories). The human colorectal cancer HCT-15 cell line (ATCC: CCL-225), called herein also “HCT-15”, was cultured in RPMI 1640 medium with 2 mM L-glutamine adjusted to contain 1.5 g/L sodium bicarbonate, 4.5 g/L glucose, 10 mM HEPES, and 1.0 mM sodium pyruvate, 1% penicillin/streptomycin, 10% fetal bovine serum.

Tumors were harvested when they had reached a minimum weight of 0.1 g. Tumor-bearing mice were anesthetized by administration of 0.02 ml/g body weight of Avertin [10 g 2,2,2-tribromoethanol (Fluka) in 10 ml 2-methyl-2-butanol (Sigma Aldrich)] intraperitoneally (i.p.).

To determine the biodistribution pattern of the europium label containing targeting agents, 4 nmol of MJ018 and 16 nmol of MJ069 were injected into the tail vein of the mice in a volume of 200 μl in physiological saline solution (Baxter). The targeting agents were allowed to circulate for 15 min. Mice were then perfused through the heart with 60 ml of physiological saline. Organs and tissues, including tumors were dissected and weighed.

For determination of the Eu content of the tumors and control or gans, 0.05-0.2 g of tissue was transferred into lysis buffer (20 mM Tris-HCl, 150 mM NaCl, 1% Nonident P40, pH 7.5) and homogenised. The Eu-content was determined from tissue lysates prepared into Enhancement solution (Perkin Elmer Wallac Ltd., Turku, Finland) and transferred to DELFIA Micro titration strip plates (Perkin Elmer Wallac Ltd., Turku, Finland). Time-resolved fluorescence was measured after incubation with a Wallac 1420 VICTOR3™ V plate reader using a D615 nm filter.

FIG. 3 shows the ratios between the Eu-content of tumors and control organs and that of muscle, after i.v. injection of the targeting agents MJ018 (black columns) and MJ069 (white columns). The tissues analysed were: A =tumor, B=heart, C=lung, D=small intestine and E=brain.

The comparison showed that the lactam-bridged MJ018 accumulated more strongly in the tumor tissue than the cystine-bridged conjugate MJ069. This indicates that the more stable lactam bridged conjugate retained its function, i.e. tumor binding, more efficiently. This finding is further pronounced by the fact that the injected amount (4 mmol) of MJ018 was four times lower than that of MJ069 (16 mmol).

### Example 13

### Plasma Protein Binding and Plasma Stability of Targeting Peptide Conjugates: Comparison of Cystine- and Lactam-Bridged Conjugates

The plasma protein binding and plasma stability of the targeting agents MJ143 (lactam-bridged; described in Example 8) and MJ142 (cystine-bridged; described below) comprising functional peptide LRSGRG was compared in mouse plasma in vitro.

The peptide conjugate MJ142 was prepared as follows:

N- and C-terminally modified peptide, acetyl-Cys*-Leu-Arg-Ser-GlyArg-Gly-Cys*-Gly-Ala-NH(CH₂CH₂O)₂CH₂CH₂NH₂, cyclic by virtue of a cystine bridge between the two cysteines (Cys*), was synthesized manually according to the general protocol described in Example 1, based on O-Bis-(aminoethyl)ethylene glycol trityl resin (Novabiochem 01-64-0285) and solid phase Fmoc-chemistry and regular protected amino acid reagents including Fmoc-Cys(Trt)OH. After Fmoc-removal treatment described above (steps 1-11 in Example 1), and capping by means of acetylation for 30 min using reagent mixture: 2 ml of acetic anhydride and 1 ml of 2,4,6-trimethylpyridine mixed in 4 ml of DMF, the resin was washed with dichloromethane, dried at argon flow and the peptide compound was cleaved off the resin, as described above in Example 1.

The peptide was cyclized in 0.05 M ammonium acetate solution at pH 8 exposed by air at room temperature over night, and then purified by RP-HPLC. The identification was based on MALDI-TOF mass spectrum. Observed positive ion M+1: 1149.53 Da. Calculated isotopic M: 1148.5 Da.

The cystine-bridged (cyclized) peptide, having C-terminal amine functionality, was labelled with indium via DTPA chelator as follows:

5.1 mg/mL (4.4 mmol/mL) of the purified peptide was stirred with 1.2 mol equivalent (5.2 mmol/mL) of ITC-DTPA, i.e. 2-(4-Isothiocyanatobenzyl)diethylenetriaminepentaacetic acid (purchased from Macrocyclics, Dallas, Tex., USA) in 0.05 M aqueous NaHCO₃ at pH 8.5 at room temperature for 17 h. 1.2 mol equivalent (5.2 mmol/mL) of InCl₃ was added and stirring was continued for 2 h. The DTPA-indium labelled product was purified by RP-HPLC at water-acetonitrile eluent gradient buffered by 0.05 M ammonium acetate. The identification of MJ142 was based on MALDI-TOF mass spectrum. Observed positive ion M+1: 1801.54 Da. Calculated isotopic M: 1823.52 Da.

### Plasma Protein Binding and Plasma Stability Assay In Vitro:

The study compounds were spiked to mouse plasma to give 10 μM concentration. The samples of 1.5 ml were incubated at 37° C. and 200 μl aliquots were taken after 0 min, 30 min, 60 min, 2 h and 4 h. Incubations were made in duplicate. The aliquot samples were ultrafiltrated for 30 min (Millipore YM-10 filters, 10 kDa cut-off, 12 000 rpm) and the supernatants were analysed using LC/MS/MS. The sample concentrations were quantified against calibration curves created using standard samples spiked to ultrafiltrated mouse plasma to concentrations 0.1, 0.5, 1, 5 and 10 μM.

LC/MS/MS Conditions:

A Finnigan Surveyor chromatographic system (Thermo-Finnigan, San Jose, Calif., USA) with autosampler, vacuum degasser and column oven was used in the analysis. The analytical column was a Waters SymmetryShield RP18, 2.1×50 mm, 3.5 μm together with Phenomenex Luna C18 precolumn, 4.0×2.0 mm, 3.0 μm (Phenomenex, Torrance, Calif., USA). The eluents were (A) 0.02% acetic acid in water (pH 3.6) and (B) methanol, with flow rate of 300 μl/min. The elution gradient was from 5% B to 90% B in five minutes, followed by column equilibration. The column oven temperature was 30° C. LC/MS/MS data was acquired with a Finnigan TSQ Discovery MAX triple quadrupole mass spectrometer equipped with an Ion MAX electrospray ionization source. Before LC/MS/MS runs, the instrument was tuned with a 5 μg/ml solution of study compounds to obtain the maximum sensitivity with multiple reaction mode. The collision gas was argon at 1.5 mTorr.

This example clearly shows that the lactam bridged conjugate stays available in plasma in greater extent for longer period than the cystine-bridged conjugate, as indicated in Table y. For both substances, about 29% (lactam)-52% (cystine) disappearance due to protein binding was detected in plasma during the 30 min ultrafiltration period used for sample prepararation before LC/MSMS analysis, even if the incubation time at 37° C. was 0 min. After 4 h incubation at 37° C. the detected amount of intact cystine-bridged conjugate was 6% of the initial amount. In comparison, still 61% of the initial amount of the lactam-bridged conjugate was intact and free after the same 4 h incubation period at 37° C.

TABLE 2
Peptide-DTPA[In] relative remaining peak areas/unbound fraction (%)

| Aliquot | 0 h | 30 min | 1 h | 2 h | 4 h |
|---|---|---|---|---|---|
| MJ142 (cystine) | 48% | 44% | 35% | 19% | 6% |
| MJ143 (lactam) | 71% | 71% | 70% | 66% | 61% |

This data shows that the lactam-bridged conjugate was greatly more stable than the cystine-bridged version, and showed considerably lower plasma protein binding.

1. A method of synthesizing a peptide conjugate comprising at least one functional peptide and at least one sulfur-linker, wherein said method comprising the steps of:
a) providing a dicarboxylic amino acid having a protecting group at its side chain carboxylic group;
b) synthesizing a peptide with a pre-determined amino acid sequence to form a functional peptide using protection, which is orthogonal with respect to the protecting group in step a);
c) constructing a synthetic peptide comprising said functional peptide of step b), said dicarboxylic amino acid of step a) C-terminally located from said functional peptide, and an N-terminal amino acid having an optionally protected non-side chain amino group;
d) removing the optional non-side chain protecting group of said N-terminal amino acid and the protecting group of the side chain of said dicarboxylic amino acid;
e) rendering the synthetic peptide obtained in step d) cyclic by connecting said non-side chain amino group to the unprotected side chain of said dicarboxylic amino acid to form a cyclic peptide; and
f) coupling a sulfur-linker, selected from the group consisting of thiols, thioethers, disulfides, sulfoxides, sulfones and thiolates, to a non-side chain carboxylic group of said dicarboxylic amino acid;
in which method said functional peptide is a peptide capable of penetrating biological membranes, organs or tissue or targeting and binding selectively to desired materials or tumor tissue and said peptide comprises 3-30 amino acids, except amino acids having a thiol group.

2. The method according to claim 1, wherein the coupling of said sulfur-linker of step f) is done to said dicarboxylic amino acid via a spacer, which is a moiety providing distance between said sulfur-linker and said cyclic peptide and/or solubility enhancing properties to the peptide conjugate.

3. The method according to claim 1, wherein the method further comprises the step of coupling an effector unit to said peptide conjugate via the sulfur containing group of said sulfur-linker.

4. The method according to claim 1, wherein step f) is performed prior to step a).

5. The method according to claim 1, wherein said N-terminal amino acid of step c) is a D-amino acid.

6. The method according to claim 5, wherein said D-amino acid is D-alanine.

7. The method according to claim 1, wherein said dicarboxylic amino acid of step a) is glutamic acid.

8. The method according to claim 1, wherein said N-terminal amino acid of step c) is a β-amino acid.

9. The method according to claim 1, wherein said dicarboxylic amino acid of step a) is aspartic acid.

10. The method according to claim 1, wherein said sulfur-linker is preloaded to a solid phase.

11. The method according to claim 10, wherein said solid phase is a peptide synthesis resin.

12. The method according to claim 1, wherein said sulfur-linker is an aminoalkanethiol.

13. The method according to claim 1, wherein said spacer is selected from the group consisting of NH—(CH₂CH₂—O)₃—CH₂—CO (Teg), NH—(CH₂CH₂—O)₃—CH₂CH₂—CO (Tegc) and Teg-Glu-Teg-Glu ((Teg-E)₂).

14. A peptide conjugate according to formula: 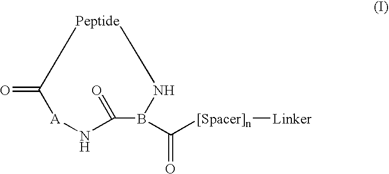
wherein
n is 0-1
“Peptide” is a functional peptide capable of penetrating biological membranes, organs or tissue or targeting and binding selectively to desired materials or tumor tissue and said peptide comprises 3-30 amino acids, except amino acids having a thiol group,
A is a residue of a D-amino acid or a β-amino acid,
B is a residue of a dicarboxylic amino acid,
“Spacer” is moiety providing distance between said sulfur-linker and said cyclic peptide and/or solubility enhancing properties to the peptide conjugate,
“Linker” is a sulfur-linker selected from the group consisting of thiols, thioethers, disulfides, sulfoxides, sulfones and thiolates, and is coupled to the non-side chain carboxylic group of B, and the C═O and NH groups denoted in the formula belong to residues A and B.

15. The peptide conjugate according to claim 14, wherein said residue of a D-amino acid is derived from D-alanine and said residue of dicarboxylic amino acid is derived from glutamic acid.

16. The peptide conjugate according to claim 14, wherein A is a residue of a β-amino acid and said residue of a dicarboxylic amino acid is derived from aspartic acid.

17. The peptide conjugate according to claim 14, wherein said sulfur-linker is an aminoalkanethiol.

18. The peptide conjugate according to claim 14, wherein said spacer is selected from the group consisting of NH—(CH₂CH₂—O)₃—CH₂—CO (Teg), NH—(CH₂CH₂—O)₃—CH₂CH₂—CO (Tegc) and Teg-Glu-Teg-Glu ((Teg-E)₂).

19. A peptide conjugate according to claim 14 for use in therapy.

20. Use of a peptide conjugate according to claim 14 for the manufacture of a tumor targeting therapeutic agent.
